(19) United States
(12) Patent Application Publication
(10) Pub. No.: US 2025/0136931 A1
(43) Pub. Date: May 1, 2025

(54) AN IMPROVED CELL CULTURE PROCESS FOR PRODUCTION OF PROTEIN

(71) Applicant: KASHIV BIOSCIENCES, LLC, Piscataway, NJ (US)

(72) Inventors: Nayan Niranjan DEVASHRI, Ahmedabad (IN); Kaumil Bhupendra bhai BHAVSAR, Ahmedabad (IN)

(21) Appl. No.: 18/855,019

(22) PCT Filed: Apr. 6, 2023
(86) PCT No.: PCT/IB2023/053519

§ 371 Date: Oct. 8, 2024

(30) Foreign Application Priority Data
Apr. 6, 2022 (IN) 202221020681

(51) Int. Cl. C12N 5/073; C07K 16/00

(52) U.S. Cl. CPC C12N 5/0604; C07K 16/00; C12N 2501/999

(57) ABSTRACT

The present invention relates to an improved cell culture process for production of proteins with the addition of mitochondria targeted antioxidant to the cell culture. The performance of the cell production is enhanced in aspects of higher viable cell density, protein titer, and reduced oxidation.

### FIELD OF THE INVENTION

The present invention relates to an improved cell culture process for production of proteins with the addition of mitochondria targeted antioxidant to the cell culture. The performance of the cell production is enhanced in aspects of higher viable cell density, protein titer, and reduced oxidation.

### BACKGROUND OF THE INVENTION

There are several techniques and methods available to enhance the product quality, which enables more efficient protein production. However, during cell culture, cells behave differently compared to in vivo in many ways. Cell culture imposes a state of oxidative stress on cells. A further problem is that cell culture media can catalyze the oxidation of compounds added to them, resulting in apparent cellular effects that are in fact due to oxidation products such as ROS.

Reactive oxygen species (ROS) are generated continuously during aerobic metabolism. ROS are highly reactive molecules and in excessive amounts, can lead to protein degradation and cell death. Accumulation of ROS within cells and/or their release into the culture media leads to lower the product quality.

Eliminating or reducing reactive oxygen species such peroxides, superoxide, hydroxyl radical and singlet oxygen has been shown to improve the cell productivity by reducing the cell death and/or apoptosis. Antioxidants may appear to have beneficial effects when added to cultured cells. Addition of antioxidants during cell culture result in reduction of reactive oxygen species and delay many events that contribute to cell death.

The present invention discloses an improved method of cell culture in bioreactor in the presence of an antioxidant compound e.g., mitochondria targeted antioxidant to maintain cell viability and increase productivity with superior quality of protein of interest.

### SUMMARY OF THE INVENTION

In an embodiment, the invention provides an improved cell culture process to culture mammalian cell to increase the protein production wherein the cells are cultured in culture medium comprising one or more mitochondria targeted antioxidant compounds.

In an embodiment, the invention provides an improved cell culture process to culture mammalian cell to increase the cell viable density wherein the cells are cultured in culture medium comprising one or more mitochondria targeted antioxidant compounds.

In an embodiment, the invention provides an improved cell culture process to culture a mammalian cell with reduce oxidation wherein the cells are cultured in culture medium comprising one or more mitochondria targeted antioxidant compounds.

In an embodiment, the invention provides the use of one or more mitochondria targeted antioxidant compounds during cell culture to increase the titer of protein of interest in comparison to the cell culture method perform without using one or more mitochondria targeted antioxidant compounds.

In an embodiment, the present invention relates to the process for enhanced production of protein. In certain embodiments, the present invention relates to the method of culturing the host cells expressing protein of interest wherein the cell culture comprises one or more mitochondria targeted antioxidant compounds which improves the production of protein of interest.

In an embodiment, the mammalian host cells are inoculated in the culture medium which is grown in a bioreactor in fed batch mode. During the production batch of the desired protein of interest, the cell culture is supplemented with one or more mitochondria targeted antioxidant compounds in order to achieve improved the cell culture characteristics.

In an embodiment, cell culture is supplemented with at least one mitochondria targeted antioxidant compound with suitable amount to achieve improved cell culture characteristics.

In certain embodiments, the mitochondria targeted antioxidant is selected from the group comprising of MitoQ, SkQ1, MitoE, and Mito-TEMPO. In preferred embodiment, the antioxidant added in the cell culture in this invention is Mito-TEMPO.

In an embodiment, the improved cell culture process for production of protein of interest, comprising:
a) culturing host cells which produce a protein of interest in cell culture under conditions that allow for protein production; and
b) supplementing one or more mitochondria targeted antioxidant compounds.
   wherein the cell culture process improves the titer of protein of interest compared to cell culture process performed without using mitochondria targeted antioxidant compounds.

In certain embodiment, the amount of antioxidant Mito-TEMPO suitable for use in cell culture comprises of at least about 0.1 nanomolar, about 0.5 nanomolar, about 1 nanomolar, about 5 nanomolar, about 10 nanomolar, about 15 nanomolar, about 20 nanomolar, about 25 nanomolar, about 30 nanomolar, about 35 nanomolar, about 40 nanomolar, about 45 nanomolar, about 50 nanomolar, about 55 nanomolar, about 60 nanomolar, about 65 nanomolar, about 70 nanomolar, about 75 nanomolar, about 80 nanomolar, about 85 nanomolar, about 90 nanomolar, about 95 nanomolar and about 100 nanomolar.

In one embodiment, the amount of antioxidant Mito-TEMPO suitable for use in cell culture comprises of at least about 0.1 nanomolar to about 95 nanomolar of Mito-TEMPO.

In one embodiment, the amount of antioxidant Mito-TEMPO suitable for use in cell culture comprises of at least about 0.5 nanomolar to about 65 nanomolar of Mito-TEMPO.

In one embodiment, the amount of antioxidant Mito-TEMPO suitable for use in cell culture comprises of at least about 1 nanomolar to about 35 nanomolar of Mito-TEMPO.

In one embodiment, the amount of antioxidant Mito-TEMPO suitable for use in cell culture comprises of at least about 1 nanomolar to about 15 nanomolar of Mito-TEMPO.

In preferred embodiment, the amount of antioxidant Mito-TEMPO suitable for use in cell culture comprises of at least about 2 nanomolar to about 7 nanomolar of Mito-TEMPO.

In an embodiment, the antioxidant Mito-TEMPO is added individually to the mammalian host cell culture. In an embodiment, the antioxidant Mito-TEMPO is added to the cell culture on daily basis or on other than a daily basis. In an embodiment, the antioxidant Mito-TEMPO is added at least about one or more times per day during the culturing period.

In an embodiment, the mitochondria targeted antioxidant compound are supplemented to the basal media, the feed media or as a bolus anytime during the cell culture process.

In an embodiment, the mitochondria targeted antioxidant compound are supplemented to the to the cell culture continuously or periodically.

In an embodiment, the antioxidant Mito-TEMPO is added every subsequent day to mammalian host cell culture during the production run of the desired protein product until the protein is harvested. In certain embodiment, the amount of Mito-TEMPO is added on about day 0, about day 1, about day 2, about day 3, about day 4, about day 5, about day 6, about day 7, about day 8, about day 9, about day 10, about day 11, about day 12, about day 13, about day 14, about day 15, about day 16, about day 17, about day 18, about day 19, about day 20, about day 21, about day 22, about day 23, about day 24, about day 25, about day 26, about day 27, about day 28, about day 29, and about day 30.

In certain embodiment, the amount of Mito-TEMPO is added every alternative day to mammalian host cell culture during the production run of the desired protein product until the protein is harvested. In preferred embodiment, the amount of Mito-TEMPO is added on about day 3, about day 5, about day 7, about day 9, about day 11, about day 13, about day 15, about day 17, about day 19, about day 21, about day 23, about day 25, about day 27, about day 29 until the day of harvest.

In an embodiment, the antioxidant Mito-TEMPO of at least about 5 nanomolar is added to mammalian host cell culture about one or more times per day on every alternative day until the day of harvest of the desired protein product.

In an embodiment, the Mito-TEMPO is supplemented in the cell culture during production run at a concentration of about 5 each on day 3, day 5, day 7, day 9, and day 11.

In an embodiment, the production of protein of interest through cell culture wherein the cell culture process improves by addition of at least about 5 nanomolar of antioxidant Mito-TEMPO wherein at least one of the cell culture characteristics is selected from the group consisting of high protein titer, high viable cell density, high cell productivity, improved sialic acid content, reduced oxidation, decreased charge-related impurities and decreased weight-related impurities.

In an embodiment, the method of increasing the protein titer wherein the cell culture process comprising addition of at least about 5 nanomolar of antioxidant Mito-TEMPO about one or more times per day on every alternative day until the day of harvest of the desired protein product whereby the protein titer is increased as compared to an identical culturing process without supplementation of Mito-TEMPO in the culture.

In an embodiment, the method of increasing the of a cell viable density wherein the cell culture process comprising addition of at least about 5 nanomolar of antioxidant Mito-TEMPO about one or more times per day on every alternative day until the day of harvest of the desired protein product whereby the cell viable density is increased as compared to an identical culturing process without supplementation of Mito-TEMPO in the culture.

In an embodiment, the method of reducing the oxidation wherein the cell culture process comprising addition of at least about 5 nanomolar of antioxidant Mito-TEMPO about one or more times per day on every alternative day until the day of harvest of the desired protein product whereby the oxidation is reduced as compared to an identical culturing process without supplementation of Mito-TEMPO in the culture.

### DETAILED DESCRIPTION OF THE INVENTION

The present invention is related to improved cell culture process for the production of desired protein of interest i.e., fusion protein or recombinant protein. The cell culture is the mammalian cell culture, in which the viable cell density and protein titer increased with reduced oxidation by the use of additives which is antioxidant when added to the cell culture. The antioxidant is added to the cell culture in an effective amount that is sufficient for maintaining the viable cell density which results in increased protein production.

The term “additive” refers to a substance or combination thereof supplied to a cell culture that produces a net increase in the yield of a biomolecule produced by the cells in the cell culture.

The term “antioxidant” refers to a compound that inhibit the release of reactive oxygen species (ROS) during aerobic metabolism which cause the oxidative damages to the cells. The cellular damages are manifested in the form of degradation of biomolecules like pigments, proteins, lipids, carbohydrates, and DNA which ultimately lead to cellular death. Antioxidant when added to culture media during cell culture enhances the viable cell density, cell growth, and protein production therein. In an embodiment the antioxidant in the cell culture results in increased protein titer, high viable cell density, decrease in weight related impurities and charge related impurities observed as compared to cell culture which lacks antioxidant.

The term “oxidation” refers to covalent modification of a protein induced either by the direct reactions with reactive oxygen species (ROS) or indirect reactions with secondary by-products of oxidative stress. Proteins are major targets for oxidation reactions, because of their rapid reaction rates with oxidants and their high abundance in cells, extracellular tissues, and body fluids. Additionally, oxidative stress is able to degrade lipids and carbohydrates to highly reactive intermediates, which eventually attack proteins at various functional sites. Oxidation of protein molecules has been shown to occur during extended cell-culture bioprocessing along with under storage and triggered by oxidative stress. During cell culture, oxidative stress has been associated with high rates of aeration, mixing speeds, temperature, osmolality, pH, and overexpression of the target protein resulting in the production of reactive oxygen species (ROS) inside the mammalian cells.

The term “Mitochondria targeted antioxidants” or “MTAs” refers to a wide range of compounds having an antioxidant group linked to a mitochondria-targeted moiety, such as triphenylphosphonium (TPP+) cation, are exemplified by TPP+-conjugated ubiquinone (MitoQ), plastoquinone (SkQ1) tocopherol, lipoic acid, spin traps, the peroxidase mimetic Ebselen, etc. MTAs are widely used in experiments for evaluating the impact of mitochondria on different pathological processes involving oxidative stress.

The term “Mito-TEMPO” refers to a mitochondria-targeted antioxidant that helps protect against oxidative damage caused mitochondria superoxide. It has superoxide and alkyl radical scavenging properties. Mito-TEMPO has been shown to facilitate in the release of proapoptotic proteins from the mitochondria, attenuating ATP-depletion recovery-mediated necrosis and apoptosis. Mito-TEMPO is a combination of the antioxidant piperidine nitroxide TEMPO with the lipophilic cation triphenylphosphonium, giving Mito-TEMPO the ability to pass through lipid bilayers with ease and accumulate several hundred-fold in mitochondria.

The term “host cell” refers to the cells are genetically modified cells which are capable to grow in cell culture medium. These cells can express recombinant proteins with post-translational modifications.

In an embodiment, of the present invention the host cell relates to “mammalian cells” which is selected from the group consisting of baby hamster kidney cells (BHK), Chinese hamster ovary cells (CHO), human kidney cells (HK), human embryonic kidney (HEK), normal fetal rhesus diploid, Vero cells and murine myeloma cells. Person ordinary skilled in the art will be aware of other host cells that may be used in accordance with these methods and compositions of the present invention.

The term “cell culture medium” refers to a solution or liquid containing nutrients designed to support the growth and survival of the cells in the culture conditions in which cells grow and produce desired protein of interest. In an embodiment, the culture medium contains one or more components such as growth factors, amino acids, vitamins, lipids, carbohydrates, chemical substances and trace elements required for the cell growth.

The term “cell culture characteristics” refers to the characteristics of cell culture observed during the culturing of cell. In an embodiment, the cell culture characteristic is selected from the group consisting of protein titer, viable cell density, cell productivity, sialic acid content, weight related impurities and charge related impurities. Person ordinary skilled in the art will be aware of other cell culture characteristics that may improve by using methods and compositions of the present invention.

The term “about”, as used herein, is intended to refer to ranges of approximately 10-20% greater than or less than the referenced value. In certain circumstances, one of skill in the art will recognize that, due to the nature of the referenced value, the term “about” can mean more or less than a 10-20% deviation from that value.

As used throughout the specification and in the appended claims, the singular forms “a,” “an,” and “the” include the plural reference unless the context clearly dictates otherwise.

The term “comprises” or “comprising” is used in the present description, it does not exclude other elements or steps. For the purpose of the present invention, the term “consisting of” is considered to be an optional embodiment of the term “comprising of”. If hereinafter a group is defined to comprise at least a certain number of embodiments, this is also to be understood to disclose a group which optionally consists only of these embodiments.

The term “viable cell density” refers to total number of cells that are surviving in the cell culture medium in a particular volume, generally per ml. The term “cell viability” refers to number of cells alive as compared to the total number of cells both dead and alive present in the culture.

The term “protein titer” refers to determining the concentration of a specific therapeutic protein produced in the cell culture. Titer is typically expressed in terms of milligrams or micrograms of protein.

The term “weight related impurities” refers to product related impurities that contribute to size heterogeneity of drug product. The formation of weight related impurities within a therapeutic fusion protein drug product as a result of protein aggregation can potentially compromise both drug efficacy and safety (e.g., eliciting unwanted immunogenic response).

The term “bioreactor” refers to a vessel in which a cell culture medium can be contained and internal conditions of which can be controlled during the culturing period.

In certain embodiment, the present invention relates to the compositions of cell culture that enhance production of a protein of interest. In certain embodiments, methods and compositions of the present invention are used to produce a recombinant proteins and fusion proteins.

In an embodiment, the present invention relates to the process for the production of protein of interest by host cell culture to achieve improved cell culture characteristics.

In an embodiment, the host cell is mammalian host cells.

In an embodiment, the improved cell culture process for production of protein of interest, comprising:
a) culturing host cells which produce a protein of interest in cell culture under conditions that allow for protein production; and
b) supplementing one or more mitochondria targeted antioxidant compounds.
   wherein the cell culture process improves the titer of protein of interest compared to cell culture process performed without using mitochondria targeted antioxidant compounds.

In an embodiment, the present invention describes an improved cell culture method or process of increasing viability and protein titer in a cell culture, comprising the addition of antioxidant Mito-TEMPO to the cell culture.

In preferred embodiment, the process of culturing cells comprising the addition of Mito-TEMPO every alternate day until the day of harvest. The addition of Mito-TEMPO in culture as provided by this method, preferably added more than one time during the culturing period more preferably once per day on an alternative day, increases, maintains viable cell density in the culture with increased protein titer and reduced oxidation, allowing the cell to grow and produce protein, thereby enhancing the protein production.

In an embodiment, the mitochondria targeted antioxidant compound are supplemented to the basal media, the feed media or as a bolus anytime during the cell culture process.

In an embodiment, the mitochondria targeted antioxidant compound are supplemented to the to the cell culture continuously or periodically.

The term “nanomolar” and “nM” are interchangeable respectively in the present invention. In certain embodiment, the amount of antioxidant Mito-TEMPO suitable for use in cell culture comprises of at least about 0.1 nanomolar, about 0.5 nanomolar, about 1 nanomolar, about 1.5 nanomolar, about 2 nanomolar, about 2.5 nanomolar, about 3 nanomolar, about 3.5 nanomolar, about 4 nanomolar, about 4.5 nanomolar, about 5 nanomolar, about 5.5 nanomolar, about 6 nanomolar, about 6.5 nanomolar, about 7 nanomolar, about 7.5 nanomolar, about 8 nanomolar, about 8.5 nanomolar, about 9 nanomolar, about 9.5 nanomolar, about 10 nanomolar, about 10.5 nanomolar, about 11 nanomolar, about 11.5 nanomolar, about 12 nanomolar, about 12.5 nanomolar, about 13 nanomolar, about 13.5 nanomolar, about 14 nanomolar, about 14.5 nanomolar, about 15 nanomolar, about 15.5 nanomolar, about 16 nanomolar, about 16.5 nanomolar, about 17 nanomolar, about 17.5 nanomolar, about 18 nanomolar, about 18.5 nanomolar, about 19 nanomolar, about 19.5 nanomolar, about 20 nanomolar, about 20.5 nanomolar, about 21 nanomolar, about 21.5 nanomolar, about 22 nanomolar, about 22.5 nanomolar, about 23 nanomolar, about 23.5 nanomolar, about 24 nanomolar, about 24.5 nanomolar, about 25 nanomolar, about 25.5 nanomolar, about 26 nanomolar, about 26.5 nanomolar, about 27 nanomolar, about 27.5 nanomolar, about 28 nanomolar, about 28.5 nanomolar, about 29 nanomolar, about 29.5 nanomolar, about 30 nanomolar, about 30.5 nanomolar, about 31 nanomolar, about 31.5 nanomolar, about 32 nanomolar, about 32.5 nanomolar, about 33 nanomolar, about 33.5 nanomolar, about 34 nanomolar, about 34.5 nanomolar, about 35 nanomolar, about 35.5 nanomolar, about 36 nanomolar, about 36.5 nanomolar, about 37 nanomolar, about 37.5 nanomolar, about 38 nanomolar, about 38.5 nanomolar, about 39 nanomolar, about 39.5 nanomolar, about 40 nanomolar, about 40.5 nanomolar, about 41 nanomolar, about 41.5 nanomolar, about 42 nanomolar, about 42.5 nanomolar, about 43 nanomolar, about 43.5 nanomolar, about 44 nanomolar, about 44.5 nanomolar, about 45 nanomolar, about 45.5 nanomolar, about 46 nanomolar, about 46.5 nanomolar, about 47 nanomolar, about 47.5 nanomolar, about 48 nanomolar, about 48.5 nanomolar, about 49 nanomolar, about 49.5 nanomolar, about 50 nanomolar, about 50.5 nanomolar, about 51 nanomolar, about 51.5 nanomolar, about 52 nanomolar, about 52.5 nanomolar, about 53 nanomolar, about 53.5 nanomolar, about 54 nanomolar, about 54.5 nanomolar, about 55 nanomolar, about 55.5 nanomolar, about 56 nanomolar, about 56.5 nanomolar, about 57 nanomolar, about 57.5 nanomolar, about 58 nanomolar, about 58.5 nanomolar, about 59 nanomolar, about 59.5 nanomolar, about 60 nanomolar, about 60.5 nanomolar, about 61 nanomolar, about 61.5 nanomolar, about 62 nanomolar, about 62.5 nanomolar, about 63 nanomolar, about 63.5 nanomolar, about 64 nanomolar, about 64.5 nanomolar, about 65 nanomolar, about 65.5 nanomolar, about 66 nanomolar, about 66.5 nanomolar, about 67 nanomolar, about 67.5 nanomolar, about 68 nanomolar, about 68.5 nanomolar, about 69 nanomolar, about 69.5 nanomolar, about 70 nanomolar, about 70.5 nanomolar, about 71 nanomolar, about 71.5 nanomolar, about 72 nanomolar, about 72.5 nanomolar, about 73 nanomolar, about 73.5 nanomolar, about 74 nanomolar, about 74.5 nanomolar, about 75 nanomolar, about 75.5 nanomolar, about 76 nanomolar, about 76.5 nanomolar, about 77 nanomolar, about 77.5 nanomolar, about 78 nanomolar, about 78.5 nanomolar, about 79 nanomolar, about 79.5 nanomolar, about 80 nanomolar, about 80.5 nanomolar, about 81 nanomolar, about 81.5 nanomolar, about 82 nanomolar, about 82.5 nanomolar, about 83 nanomolar, about 83.5 nanomolar, about 84 nanomolar, about 84.5 nanomolar, about 85 nanomolar, about 85.5 nanomolar, about 86 nanomolar, about 86.5 nanomolar, about 87 nanomolar, about 87.5 nanomolar, about 88 nanomolar, about 88.5 nanomolar, about 89 nanomolar, about 89.5 nanomolar, about 90 nanomolar, about 90.5 nanomolar, about 91 nanomolar, about 91.5 nanomolar, about 92 nanomolar, about 92.5 nanomolar, about 93 nanomolar, about 93.5 nanomolar, about 94 nanomolar, about 94.5 nanomolar, about 95 nanomolar, about 95.5 nanomolar, about 96 nanomolar, about 96.5 nanomolar, about 97 nanomolar, about 97.5 nanomolar, about 98 nanomolar, about 98.5 nanomolar, about 99 nanomolar, about 99.5 nanomolar, and about 100 nanomolar.

In certain embodiment, the amount of antioxidant Mito-TEMPO suitable for use in cell culture comprises of at least about 1 nanomolar to about 1000 micromolar. In certain embodiment, the amount of antioxidant Mito-TEMPO suitable for use in cell culture comprises of at least 1 nanomolar to about 100 nanomolar. In certain embodiment, the amount of antioxidant Mito-TEMPO suitable for use in cell culture comprises of at least 100 nanomolar to about 200 nanomolar. In certain embodiment, the amount of antioxidant Mito-TEMPO suitable for use in cell culture comprises of at least 200 nanomolar to about 300 nanomolar. In certain embodiment, the amount of antioxidant Mito-TEMPO suitable for use in cell culture comprises of at least 300 nanomolar to about 400 nanomolar. In certain embodiment, the amount of antioxidant Mito-TEMPO suitable for use in cell culture comprises of at least 400 nanomolar to about 500 nanomolar. In certain embodiment, the amount of antioxidant Mito-TEMPO suitable for use in cell culture comprises of at least 500 nanomolar to about 600 nanomolar. In certain embodiment, the amount of antioxidant Mito-TEMPO suitable for use in cell culture comprises of at least 600 nanomolar to about 700 nanomolar. In certain embodiment, the amount of antioxidant Mito-TEMPO suitable for use in cell culture comprises of at least 700 nanomolar to about 800 nanomolar. In certain embodiment, the amount of antioxidant Mito-TEMPO suitable for use in cell culture comprises of at least 800 nanomolar to about 900 nanomolar. In certain embodiment, the amount of antioxidant Mito-TEMPO suitable for use in cell culture comprises of at least 900 nanomolar to about 1 micromolar. In certain embodiment, the amount of antioxidant Mito-TEMPO suitable for use in cell culture comprises of at least 1 micromolar to about 100 micromolar. In certain embodiment, the amount of antioxidant Mito-TEMPO suitable for use in cell culture comprises of at least 100 micromolar to about 200 micromolar. In certain embodiment, the amount of antioxidant Mito-TEMPO suitable for use in cell culture comprises of at least 200 micromolar to about 300 micromolar. In certain embodiment, the amount of antioxidant Mito-TEMPO suitable for use in cell culture comprises of at least 300 micromolar to about 400 micromolar. In certain embodiment, the amount of antioxidant Mito-TEMPO suitable for use in cell culture comprises of at least 400 micromolar to about 500 micromolar. In certain embodiment, the amount of antioxidant Mito-TEMPO suitable for use in cell culture comprises of at least 600 micromolar to about 600 micromolar. In certain embodiment, the amount of antioxidant Mito-TEMPO suitable for use in cell culture comprises of at least 600 micromolar to about 700 micromolar. In certain embodiment, the amount of antioxidant Mito-TEMPO suitable for use in cell culture comprises of at least 700 micromolar to about 800 micromolar. In certain embodiment, the amount of antioxidant Mito-TEMPO suitable for use in cell culture comprises of at least 800 micromolar to about 900 micromolar. In certain embodiment, the amount of antioxidant Mito-TEMPO suitable for use in cell culture comprises of at least 900 micromolar to about 1000 micromolar.

In one embodiment, the amount of antioxidant Mito-TEMPO suitable for use in cell culture comprises of at least about 0.1 nanomolar to about 95 nanomolar of Mito-TEMPO.

In one embodiment, the amount of antioxidant Mito-TEMPO suitable for use in cell culture comprises of at least about 0.5 nanomolar to about 65 nanomolar of Mito-TEMPO.

In one embodiment, the amount of antioxidant Mito-TEMPO suitable for use in cell culture comprises of at least about 1 nanomolar to about 35 nanomolar of Mito-TEMPO.

In one embodiment, the amount of antioxidant Mito-TEMPO suitable for use in cell culture comprises of at least about 1 nanomolar to about 15 nanomolar of Mito-TEMPO.

In preferred embodiment, the amount of antioxidant Mito-TEMPO suitable for use in cell culture comprises of at least about 2 nanomolar to about 7 nanomolar of Mito-TEMPO.

In preferred embodiment, the antioxidant Mito-TEMPO of at least about 5 nanomolar is added to mammalian host cell culture about once per day on every alternative day until the day of harvest which enhanced the protein titer, viable cell density, and reduced oxidation when compared to cell culture which lacks antioxidant Mito-TEMPO.

In an embodiment, the Mito-TEMPO is supplemented in the cell culture during production run at a concentration of about 5 nM each on day 3, day 5, day 7, day 9, and day 11.

In an embodiment, the protein of interest is selected from Etanercept, Abatacept, Rituximab, Palivizumab, Infliximab, Trastuzumab, Alemtuzumab, Adalimumab, Ibritumomab, Omalizumab, Cetuximab, Bevacizumab, Natalizumab, Eculizumab, Certolizumab pegol, Ustekinumab, Canakinumab, Golimumab, Ofatumumab, Tocilizumab, Denosumab, Belimumab, Ipilimumab, Brentuximab vedotin, Pertuzumab, Trastuzumab emtansine, Raxibacumab, Obinutuzumab, Siltuximab, Ramucirumab, Vedolizumab, Nivolumab, Pembrolizumab, Darucizumab, Necitumumab, Dinutuximab, Secukinumab, Mepolizumab, Alirocumab, Evolocumab, Daratumumab, Elotuzumab, Ixekizumab, Reslizumab, Olaratumab, Bezlotoxumab, Atezolizumab, Obiltoxaximab, Sarilumab, Ocrelizumab, Tildrakizumab, Romosozumab, Brolucizumab, Crizanlizumab.

The present invention provides an example for illustration purpose which should not be considered to limit the scope of the present invention with the described examples.

### Example 1

### Process for Improved Composition of Fusion Protein Production (CTLA4Ig)

The cell bank vial was thawed in Shake flask 125 (SF125) into the seed media and cell counts were checked. The incubation was continued for 3-5 days during which cells reached the optimum cell density for subculture. The cells were subsequently expanded in Shake flask 500 (SF500) to generate sufficient inoculum for Ambr®250 bioreactors (250 mL).

Production Fed batch bioreactor run was executed at Ambr®250 bioreactor using process parameters as mentioned below.

Control Process

| Sr. | | | Setpoints/parameter |
|---|---|---|---|
| No | Process Parameter | Unit | details |
| 1 | Post Inoculation Initial | mL | 190 |
| | volume | | |
| 2 | DO % | % Saturation | 60 |
| 3 | pH | N/A | 7.0 ± 0.15 |
| 4 | Temperature | ° C. | 37.0 |
| 5 | Agitation rate | RPM | 550-900 |
| 7 | Initial seeding cell | ×10⁶ cells/mL | 0.3 |
| | density | | |
| 10 | Antifoam | % | Up to 10% addition (as |
| | | | required) |
| 11 | % DO maintenance by | mL/min | On demand (cascade) |
| | Air + O2 | | |
| 12 | Air flow. | mL/min | 0-5 |
| 13 | O₂ flow | mL/min | 0-25 |
| 14 | Air overlay flow rate | mL/min | 2.5 |
| 15 | Harvest Criteria | N/A | Day 12 or Viability |
| | | | 60 ± 10% |
| 16 | MitoTempo | nM | 5 nM each on Day 3, 5, |
| | | | 7, 9, 11 (Total 25 nM) |

The standard fed-batch culture process involved cell culturing for 12 days and was harvested depending upon the culture cell viability.

When the control process was spiked with antioxidant Mito Tempo (total 25 nM added throughout the culturing period) 5 nM each on day 3, 5, 7, 9, 11. It was observed that the harvested protein titer was improved by 28% when analysed by Protein-A HPLC method. It was also observed that overall oxidation of the product was decreased by 18% when analysed by LC-MS method. The overall peak viable cell density of the culture was also found to have increased by 11%.

| | | Control + |
|---|---|---|
| | | MitoTempo |
| Parameter | Control Process | Process |
| Peak cell density by Automated | 16.9 | 18.8 |
| cell counter | | |
| (×10⁶ cells/mL) | | |
| Harvest Titer by Protein-A HPLC | 2.1 | 2.7 |
| (g/L) | | |
| Harvest Oxidation (%) by LC-MS | 14.9 | 12.2 |

### Example 2

### Process for Improved Composition of Antibody Production.

The cell bank vial is thawed in Shake flask 125 (SF125) into the seed media and cell counts are checked. The incubation is continued for 3-5 days during which cells reached the optimum cell density for subculture. The cells are subsequently expanded in Shake flask 500 (SF500) to generate sufficient inoculum for Ambr®250 bioreactors (250 mL).

Production Fed batch bioreactor run is executed at Ambr®250 bioreactor using process parameters as mentioned below.

Control Process

| Sr. | | | Setpoints/parameter |
|---|---|---|---|
| No | Process Parameter | Unit | details |
| 1 | Post Inoculation Initial | mL | 190 |
| | volume | | |
| 2 | DO % | % Saturation | 60 |
| 3 | pH | N/A | 7.0 ± 0.15 |
| 4 | Temperature | ° C. | 37.0 |
| 5 | Agitation rate | RPM | 550-900 |
| 7 | Initial seeding cell density | ×10⁶ cells/mL | 0.3 |
| 10 | Antifoam | % | Up to 10% addition (as |
| | | | required) |
| 11 | % DO maintenance by | mL/min | On demand (cascade) |
| | Air + O2 | | |
| 12 | Air flow. | mL/min | 0-5 |
| 13 | O₂ flow | mL/min | 0-25 |
| 14 | Air overlay flow rate | mL/min | 2.5 |
| 15 | Harvest Criteria | N/A | Day 12 or Viability |
| | | | 60 ± 10% |
| 16 | MitoTempo | nM | 5 nM each on Day 3, |
| | | | 5, 7, 9, 11. |

The standard fed-batch culture process involved cell culturing for 12 days and it is harvested depending upon the culture cell viability.

When the control process is spiked with antioxidant MitoTempo (total 25 nM added throughout the culturing period) 5 nM each on day 3, 5, 7, 9, 11. It is observed that the harvested protein titer is improved when analysed by Protein-A HPLC method. It is also observed that overall oxidation of the product is decreased when analysed by LC-MS method. The overall peak viable cell density of the culture is also found to have increased.

We claim:

1. An improved cell culture process for production of protein of interest, comprising:
a) culturing host cells which produce a protein of interest in cell culture under conditions that allow for protein production; and
b) supplementing one or more mitochondria targeted antioxidant compounds.
wherein the cell culture process improves the titer of protein of interest compared to cell culture process performed without using mitochondria targeted antioxidant compounds.

2. The process as claimed in claim 1, wherein the cell culture process improved cell viable density.

3. The process as claimed in claim 1, wherein the cell culture process reduces oxidation of the protein of interest.

4. The process as claimed in claim 1, the mitochondria targeted antioxidant compounds are selected from MitoQ, SkQ1, MitoE, and Mito-TEMPO.

5. The process as claimed in claim 1, wherein the host cell is selected from CHO, myeloma, and COS cells.

6. The process as claimed in claim 5, wherein the host cell is CHO cells.

7. The process as claimed in claim 1, wherein the mitochondria targeted antioxidant compounds are maintained in the cell culture at a concentration of about 0.1 nM to about 100 nM.

8. The process as claimed in claim 1, wherein the mitochondria targeted antioxidant compounds are maintained in the cell culture at a concentration of about 0.5 nM to about 65 nM.

9. The process as claimed in claim 1, wherein the mitochondria targeted antioxidant compounds are maintained in the cell culture at a concentration of about 1 nM to about 35 nM.

10. The process as claimed in claim 1, wherein the mitochondria targeted antioxidant compounds are maintained in the cell culture at a concentration of about 1 nM, about 2 nM, about 3 nM, about 4 nM, about 5 nM, about 6 nM, about 7 nM, about 8 nM, about 9 nM, about 10 nM, about 15 nM.

11. The process as claimed in claim 1, wherein the mitochondria targeted antioxidant compound are supplemented to the basal media, the feed media or as a bolus anytime during the cell culture process.

12. The process as claimed in claim 1, wherein the mitochondria targeted antioxidant compounds are supplemented to the cell culture continuously or periodically.

13. The process as claimed in claim 1, wherein the mitochondria targeted antioxidant compounds are supplemented to the cell culture on every alternative day during production run until the protein is harvested.

14. The process as claimed in claim 1, wherein the mitochondria targeted antioxidant compounds are supplemented to the cell culture about one or more times per day on every alternative day during production run until the protein is harvested.

15. The process as claimed in claim 4, wherein the mitochondria targeted antioxidant compound is Mito-TEMPO and is supplemented to the cell culture about one or more times per day on every alternative day during production run until the protein is harvested.

16. The process as claimed in claim 1, wherein the Mito-TEMPO is supplemented in the cell culture during production run at a concentration of about 5 nM each on day 3, day 5, day 7, day 9, and day 11.

17. The process as claimed in claim 1, wherein the protein of interest is selected from Etanercept, Abatacept, Rituximab, Palivizumab, Infliximab, Trastuzumab, Alemtuzumab, Adalimumab, Ibritumomab, Omalizumab, Cetuximab, Bevacizumab, Natalizumab, Eculizumab, Certolizumab pegol, Ustekinumab, Canakinumab, Golimumab, Ofatumumab, Tocilizumab, Denosumab, Belimumab, Ipilimumab, Brentuximab vedotin, Pertuzumab, Trastuzumab emtansine, Raxibacumab, Obinutuzumab, Siltuximab, Ramucirumab, Vedolizumab, Nivolumab, Pembrolizumab, Darucizumab, Necitumumab, Dinutuximab, Secukinumab, Mepolizumab, Alirocumab, Evolocumab, Daratumumab, Elotuzumab, Ixekizumab, Reslizumab, Olaratumab, Bezlotoxumab, Atezolizumab, Obiltoxaximab, Sarilumab, Ocrelizumab, Tildrakizumab, Romosozumab, Brolucizumab, Crizanlizumab.
